(19) United States
(12) Patent Application Publication
(10) Pub. No.: US 2021/0322784 A1
(43) Pub. Date: Oct. 21, 2021

(54) TISSUE TREATMENT DEVICE AND METHOD OF USING THE SAME

(71) Applicant: Ellor LLC, Palm Beach Gardens, FL (US)

(72) Inventors: David SHUTER, Palm Beach Gardens, FL (US); Daniel SHUTER, New York, NY (US)

(73) Assignee: Ellor LLC, Palm Beach Gardens, FL (US)

(21) Appl. No.: 17/236,357

(22) Filed: Apr. 21, 2021

Related U.S. Application Data
(60) Provisional application No. 63/013,245, filed on Apr. 21, 2020.
(60) Provisional application No. 63/087,616, filed on Oct. 5, 2020.

(51) Int. Cl. A61N 5/06

(52) U.S. Cl. CPC A61N 5/0616; A61N 2005/0626; A61N 2005/0666; A61N 2005/0651; A61N 2005/0647

(57) ABSTRACT

A phototherapy device comprising a plurality of light sources adapted for emitting at a treatment wavelength and a contact layer, wherein the contact layer is adapted to retain a treatment fluid, the plurality of light sources are electrically isolated from treatment fluid retained by the contact layer, and the plurality of light sources are coupled to the contact layer. The plurality of light sources can be releasably coupled or permanently coupled to the contact layer.

A phototherapy kit comprising the phototherapy device and instructions for selection or placement of the plurality of lights, or for selection of settings for the plurality of lights, on phototherapy device. The phototherapy device or kit can also include a liquid treatment absorbed in the contact layer or to be applied to the contact layer.

A method of treating tissue, comprising applying electromagnetic radiation to tissue using the phototherapy device.

### CROSS-REFERENCE TO RELATED APPLICATIONS

This application claims priority under 35 U.S.C. § 119(e) to U.S. Provisional Application No. 63/013,245, filed Apr. 21, 2020, and U.S. Provisional Application No. 63/087,616, filed Oct. 5, 2020, the entireties of which are incorporated herein by reference.

### TECHNICAL FIELD

The disclosure relates to generally to devices and methods for the treatment of tissue, such as skin, and more particularly to phototherapy devices and methods of using such devices.

### BACKGROUND

Phototherapy uses light to treat certain skin conditions. A variety of phototherapy devices have been used for application in a professional setting, such as doctor's offices, or in an at-home setting. However, such devices can be bulky, inefficient, or otherwise impractical to store, set up and/or use. There is still room for improvements to phototherapy devices and their applications.

### SUMMARY

A phototherapy device is described herein. The device includes a plurality of light sources adapted for emitting at a treatment wavelength and a contact layer adapted to retain a treatment fluid. The plurality of light sources are electrically isolated from treatment fluid retained by the contact layer. The plurality of light sources can be coupled to the contact layer. In some embodiments, the plurality of light sources can be releasably coupled to the contact layer. In other embodiments, the plurality of light sources can be permanently coupled to the contact layer.

In some embodiments, the phototherapy device can be pliable, including when the plurality of light sources are coupled to the contact layer.

In some embodiments, the plurality of light sources can coupled to the contact layer by a backing layer, one or more backings, or one or more capsules. In such embodiments, the plurality of lights can be attached to the backing layer, one or more backings, or one or more capsules; and the backing layer, backings, or capsules can be coupled to the contact layer.

In some embodiments, phototherapy device can also include a reflective layer adapted for reflecting the treatment wavelength toward the contact layer. The reflective layer can be included on the backing layer, backings, capsules, or any portions thereof. In some embodiments, the reflective layer can be adapted to reflect and distribute energy in the treatment wavelength and provide photon recycling for energy not initially absorbed by tissue of a wearer.

In some embodiments wherein the plurality of lights can be releasably coupled to the contact layer, one or both of the plurality of lights and the contact layer can include one or more retaining members for releasably engaging with the other.

In some embodiments, the plurality of light sources comprise one or more retaining members for releasably engaging with the contact layer. In some embodiments, the backings or capsules for the plurality of light sources can include the retaining members for releasably engaging with the contact layer.

In some embodiments, the contact layer comprises one or more retaining members for releasably engaging with one or more of the plurality of light sources. In some embodiments, the one or more retaining members can be coupled to the contact layer in fixed locations corresponding to one or more treatment areas on an intended wearer. In some embodiments, the contact layer can be impregnated, coated or covered with the one or more retaining members.

In some embodiments, the contact layer includes an absorbent layer adapted for retaining the treatment fluid. In some embodiments, a back surface of the contact layer can include a sealing layer of a liquid impermeable material.

In some embodiments, the backings or capsules for the plurality of light sources, or portions thereof, can be liquid impermeable (e.g. forming a water-tight volume).

In some embodiments including the backing layer, the plurality of light sources are between the backing layer and the contact layer. In some embodiments, at least a portion of the backing layer and the contact layer can be permanently coupled together. In some embodiments, the backing layer and the contact layer can be releasably coupled together.

In some embodiments, the backing layer can be liquid impermeable and the contact layer includes a sealing layer of a liquid impermeable material and an absorbent layer adapted for retaining the treatment fluid. The backing layer and the sealing layer can form a waterproof enclosure around the plurality of light sources.

In some embodiments, the contact layer can be adapted for transmitting the treatment wavelength. In some embodiments, at least 10% of light in the treatment wavelength can be transmitted through the contact layer. In some embodiments, the contact layer can include a plurality of light windows adapted for transmitting the treatment wavelength.

In some embodiments, one or more of the plurality of light sources is an LED. In some embodiments, each of the plurality of light sources is an LED.

In some embodiments, the plurality of light sources can be adapted so that at least one of the following is true: (i) they can emit at more than one target wavelength, (ii) they emit at more than one target intensity, (iii) they can emit either continuously or pulsed intermittently, and (iv) they can emit for more than one pre-programmed duration.

In some embodiments, the treatment wavelength is in the range of 400 to 490 nm or 620 to 1700 nm.

In some embodiments, the phototherapy device also includes a power source for powering the plurality of light sources.

In some embodiments, the phototherapy device also includes a communication component adapted for transmitting data and receiving data with a remote device.

In some embodiments, the phototherapy device is adapted so that (i) the phototherapy device it can be folded in half, (ii) the phototherapy device can assume a flat configuration when placed in a single layer on a flat surface, or (iii) both options (i) and (ii).

In some embodiments, the phototherapy device also includes a treatment fluid. In some embodiments, the contact layer is adapted to contact a wearer of the phototherapy device and the contact layer is adapted to deliver treatment fluid retained therein to the wearer of the phototherapy device. The treatment fluid can be provided absorbed within the contact layer, or can be provided in a package containing the liquid treatment to be applied to the contact layer.

In some embodiments, the treatment liquid can include an ingredient selected from the group consisting of retinol, salicylic acid, zinc, glucolics, tea tree oil, vitamin C, kojic acid, peptides, growth factors, and antioxidants, niacinamide, polyphenols, glutathione, flavonoids, stem cells, primrose, grape seed extract, vitamin E, co-Q10, alpha hydroxy acids, beta hydroxy acids, licorice, grapefruit, ferulic acid, glycolic acid, citric acid, zinc, green tea, cucumber, thyme, glycerin, argan oil, hyaluronic acid, aloe vera, ceramides, rosehip oil, sea kelp, jojoba, lactic acid, fruit/plant enzymes, resveratrol, antioxidants, omega 3′s, essential oils, seaweed, and noni.

In some embodiments, the phototherapy device can be provided in a tissue treatment package. The package can include a phototherapy device described herein and a liquid treatment. In some embodiments, the package includes a sealed package, wherein the phototherapy device is contained within the sealed package, and wherein: (i) a liquid treatment is absorbed in the contact layer of the phototherapy device, or (ii) the sealed package contains a liquid treatment to be applied to the contact layer.

In some embodiments, the phototherapy device is contained within the package is a folded phototherapy device. In some embodiments, the phototherapy device contained within the package phototherapy device can assume a flat configuration.

In some embodiments, the phototherapy device and/or the tissue treatment package can be provided in a phototherapy kit. The kit can include a phototherapy device described herein and instructions for selection or placement of the plurality of lights, or for selection of settings for the plurality of lights, on phototherapy device.

In some embodiments, the kit can also include a plurality of two or more different types of lights.

A method of treating tissue is also described herein. The method includes applying electromagnetic radiation to tissue using a phototherapy device as described herein.

In some embodiments, the method can also include receiving a selection of one or more of the following: (i) a target wavelength, (ii) an emission intensity, (iii) application of the treatment wavelength as either continuously or pulsed, and (iv) application of the treatment wavelength for one of at least two available durations.

In some embodiments, the phototherapy device is adapted to be worn on a part of the body selected from the group consisting of the face, under the eye, lips, breasts, décolletage, neck, legs, arms, hands, feet, abdomen, back, and groin.

### BRIEF DESCRIPTION OF THE DRAWINGS

The features and advantages of the present disclosures will be more fully disclosed in, or rendered obvious by the following detailed descriptions of example embodiments. The detailed descriptions of the example embodiments are to be considered together with the accompanying drawings wherein like numbers refer to like parts and further wherein:

FIG. 1 illustrates a cross-sectional view of a phototherapy device as disclosed herein.

FIG. 2 illustrates a cross-sectional view of a phototherapy device including light windows as disclosed herein.

FIG. 3 illustrates a cross-section of one configuration of a backing layer as described herein.

FIG. 4 illustrates a cross-section of one configuration of a backing layer as described herein.

FIG. 5 illustrates the backing layer, contact layer and battery of an embodiment as described herein.

FIG. 6 illustrates the backing layer, contact layer and battery of another embodiment as described herein.

FIG. 7 illustrates the backing layer, contact layer and battery of yet another an embodiment as described herein.

FIG. 8 is a bottom view of a phototherapy mask as described herein.

FIG. 9 is a top view of the phototherapy mask of FIG. 8.

FIG. 10 is a bottom view of the phototherapy mask of FIG. 8 where the plurality of lights have been activated.

FIG. 11 is a cross-sectional view of one construction of the phototherapy mask of FIG. 10 taken along cut line A-A.

FIG. 12 is a cross-sectional view of an alternate construction of the phototherapy mask of FIG. 10 taken along cut line A-A.

FIG. 13 illustrates the backing layer, contact layer and battery for a disposable embodiment of the phototherapy mask described herein.

FIG. 14 illustrates the backing layer, contact layer and battery for a partially disposable embodiment of the phototherapy mask described herein.

FIG. 15 illustrates the backing layer, contact layer and battery for another partially disposable embodiment of the phototherapy mask described herein.

FIG. 16 illustrates the backing layer, contact layer and battery for a multi-use embodiment of the phototherapy mask described herein.

FIG. 17 illustrates a top view of a phototherapy device adapted for treating under a wearer's eye.

FIG. 18 illustrates a bottom view of phototherapy devices adapted for treating the lips of a wearer.

FIG. 19 illustrates top (left) and bottom (right) views of phototherapy device adapted for treating a breast of a wearer.

FIG. 20A and 20B illustrate top views of phototherapy devices adapted for treating the genital area of female and male wearer's respectively.

FIG. 21 illustrates a bottom view of phototherapy devices adapted for treating the neck of a wearer.

FIG. 22 illustrates a perspective views of phototherapy devices adapted for treating the hands (left) and feet (right) of a wearer.

FIG. 23 illustrates a top view of phototherapy devices adapted for treating the décolletage of a wearer.

FIG. 24 illustrates a bottom (left) and top (right) views of rectangular and round phototherapy devices adapted for treating a wearer.

FIG. 25 illustrates a tissue treatment package containing a folded phototherapy device as described herein.

FIG. 26 illustrates a cross-sectional view of the tissue treatment package of FIG. 25 taken along cut line B-B.

FIG. 27 illustrates a mask that includes straps secured by hook and loop material as described herein.

FIG. 28 illustrates a mask that includes straps secured by a button and button hole as described herein.

FIG. 29 illustrates a mask that includes straps secured by a slot and adhesive as described herein.

FIG. 30 illustrates a mask that includes straps secured by a button as described herein.

FIG. 31 illustrates a cross-sectional view of a phototherapy device as described herein.

FIG. 32 illustrates a top view of a phototherapy device as described herein.

FIG. 33 illustrates a cross-sectional view of a phototherapy device as described herein.

FIG. 34 illustrates a top view of a phototherapy device as described herein.

FIG. 35A illustrates a cross-sectional view of a phototherapy device as described herein.

FIG. 35B illustrates a cross-sectional view of a phototherapy device as described herein.

FIG. 35C illustrates a cross-sectional view of a phototherapy device as described herein.

FIG. 35D illustrates a cross-sectional view of a phototherapy device as described herein.

FIG. 36 illustrates a top view of a phototherapy device as described herein.

FIG. 37A illustrates a bottom view of a plurality of lights described herein.

FIG. 37B illustrates a bottom view of a plurality of lights described herein.

FIG. 38 illustrates a perspective view of a phototherapy kit as described herein.

FIG. 39 illustrates a top view of a phototherapy device as described herein as described herein.

FIG. 40 illustrates a bottom view of a plurality of lights described herein as described herein.

FIG. 41 illustrates a cross-sectional view of a phototherapy device as disclosed herein as described herein.

FIG. 42 illustrates a cross-sectional view of a phototherapy device as disclosed herein as described herein.

### DETAILED DESCRIPTION

The description of the preferred embodiments is intended to be read in connection with the accompanying drawings, which are to be considered part of the entire written description of these disclosures. It should be understood, however, that the present disclosure is not intended to be limited to the particular forms disclosed. Rather, the present disclosure covers all modifications, equivalents, and alternatives that fall within the spirit and scope of these exemplary embodiments.

In this description, relative terms such as “horizontal,” “vertical,” “up,” “down,” “top,” “bottom,” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. The terms “couple,” “coupled,” “operatively coupled,” “operatively connected,” and the like should be broadly understood to refer to connecting devices or components together either mechanically, electrically, wired, wirelessly, or otherwise, such that the connection allows the pertinent devices or components to operate (e.g., communicate) with each other as intended by virtue of that relationship.

The device includes a plurality of light sources 14 adapted for emitting at a treatment wavelength and a contact layer 16. The contact layer 16 is a sheet of material adapted to contact the tissue of a wearer (W). The contact layer 16 is adapted to retain a treatment fluid. The plurality of lights 14 are electrically isolated from treatment fluid retained by the contact layer 16. The plurality of lights 14 can be releasably or permanently coupled to the contact layer 16. In some embodiments, the plurality of lights 14 can be coupled to the contact layer 16 by a backing layer 12 or by one or more backings or capsules 15.

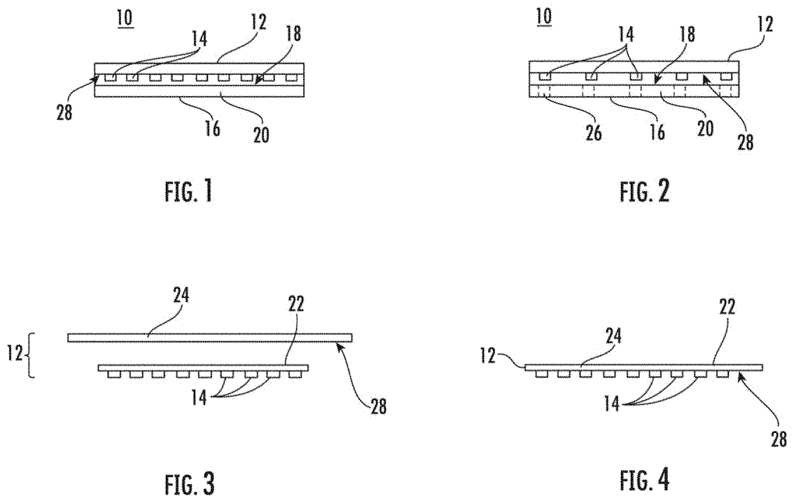

Turning to the drawings, FIGS. 1 and 2 show a cross-section of a phototherapy device 10 that includes a backing layer 12, a plurality of lights 14, and a contact layer 16. The plurality of lights 14 are between the backing layer 12 and the contact layer 16, and the plurality of lights 14 are electrically isolated from treatment fluid retained by the contact layer 16. In such embodiments, as shown in FIGS. 11 and 12, the plurality of lights 14 can be contained within a water-tight volume 34.

In particular, the backing layer 12 and contact layer 16 can be coupled together in a manner that treatment fluid absorbed in the contact layer 16 cannot reach and short out the plurality of lights 14 or conduct electricity from the plurality of light (or an associated power source) to shock the wearer of the phototherapy device 10. In some embodiments, as best shown in FIGS. 11 and 12, the backing layer 12 and contact layer 16 can be coupled together to form a water-tight seal.

The phototherapy device 10 is adapted so that the contact layer 16 is applied to the tissue of a wearer. In this manner, treatment fluid absorbed stored in the contact layer contacts the tissue of the wearer while the treatment wavelength is simultaneously applied to the tissue. This combination of the treatment fluid with the treatment wavelength can treat, prevent, or slow a wide variety of tissue conditions including, but not limited to, acne, discoloration, dryness, ageing, wrinkles, fine lines, infections, wounds (open or healing), and scars. As will be understood, tissue is intended to reference external tissue of a wearer/user, including the skin, including wounds, both open and in any stage of healing.

In some embodiments, the phototherapy device is pliable. In particular, the phototherapy device can adapt to the topography of the portion of the wearer's body it is placed upon. In some embodiments, the phototherapy is adapted to be folded in half. In some embodiments, the phototherapy device 10 can assume a flat configuration when placed in a single layer on a flat surface.

As shown in FIGS. 1-5, 7, 11, and 12, in some embodiments, the backing layer 12 comprises a reflective layer 28 adapted for reflecting the treatment wavelength toward the contact layer 16. In some embodiments, the reflective layer 28 is adapted to distribute energy at the treatment wavelength and provide photon recycling for energy not initially absorbed by tissue of a wearer. For example, photon recycling can be enhanced by reflecting photons at the treatment wavelength traveling away from the tissue back towards the tissue of the wearer. The reflective layer 28 can be smooth, patterned, or irregular and can be adapted to minimize “hot spots” and allow photon energy to be absorbed throughout the targeted anatomic structure.

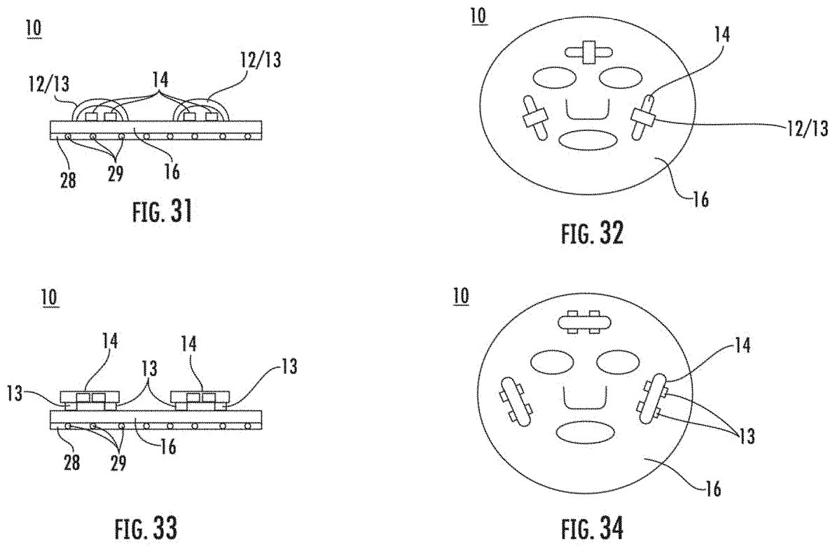

As shown in FIGS. 31 and 33, in some embodiments, the treatment fluid comprises an alternate or additional reflective layer 28, wherein the treatment fluid can include reflective particles 29 adapted for reflecting or refracting the treatment wavelength toward the tissue of the wearer. The reflective particles 29 are preferably non-toxic and suitable for cosmetic application. The reflective particles 29 are a suitable particle size for dispersion in the treatment fluid. For example in some embodiments, the reflective particles 29 can be 0.1 mm or less, 0.15 mm or less, 0.2 mm or less, 0.3 mm or less, 0.5 mm or less, 1 mm or less, 2 mm or less, 5 mm or less, 6 mm or less, or 7 mm or less. In some embodiments, the particles 29 can range between any two of the foregoing values, including values encompassed therein (e.g., 0.3 mm or less includes 203.3 microns or less, 101.6 microns or less, etc.). For example, the particle 29 size can range from about 0.1-5 mm, or 0.1-0.3 mm, or 1-6.25 mm.

In some embodiments, the reflective particles 29 can include, for example, ingredients or coatings comprising ingredients selected from acrylic, polyester, such as polyurethane terephthalate (PET), glass, stone, such as mica or malachite, paper, plastic, metal, materials from insects (e.g., exoskeleton), or other suitable reflective materials. In some embodiments, the reflective particles 29 are adapted to distribute energy at the treatment wavelength and provide photon recycling for energy not initially absorbed by tissue of a wearer. For example in some embodiments, the reflective particles 29 can reflect or bounce photons at the treatment wavelength traveling away from the tissue back towards the tissue of the wearer.

In some embodiments, the reflective particles 29 can include a suitable shape and size or materials that allow the light to diverge. In some embodiments, the reflective particles 29 can refract photons at the treatment wavelength traveling towards the tissue of the wearer to better spread the light to the tissue. In some embodiments, the particles 29 can have a refractive index between 1.4-1.6, and in some embodiments, the transmission at 635 nm can be >70%. In some embodiments, the reflective particles 29 can include a combination of reflecting and refracting particles.

In some embodiments, the contact layer 16 is adapted for transmitting the treatment wavelength so that the treatment wavelength can pass from the light sources 14 through the contact layer 16 to the wearer of the phototherapy device 10. In some embodiments, the component(s) of the contact layer 16 allow at least 10% of light in the treatment wavelength to transmit through the contact layer 16. In some embodiments, at least 20%, at least 30%, at least 40%, at least 50%, at least 60%, at least 70%, at least 80%, or at least 90% of light at the treatment wavelength to transmit through the contact layer 16. In some embodiments, at least 20%, at least 30%, at least 40%, at least 50%, at least 60%, at least 70%, at least 80%, or at least 90% of light at the treatment wavelength to transmit through the contact layer 16 when the treatment fluid is absorbed in the contact layer 16.

In some embodiments, as shown in FIGS. 2. 5-7, 18, 19, 21, and 24, the contact layer 16 includes a plurality of light windows 26, which are adapted to allow at least 60% of light in the treatment wavelength to transmit through them. In some embodiments, at least 70%, at least 80%, at least 90%, or at least 95% of light in the treatment wavelength transmits through the light windows 26.

In some embodiments, as shown in FIGS. 1 and 2, the contact layer 16 can include a sealing layer 18 and an absorbent layer 20. In some embodiments, the sealing layer 20 can be impermeable to the treatment fluid and the absorbent layer 20 can be on the side of the phototherapy device that contacts the wearer's tissue. In some embodiments, as shown in FIGS. 2 and 5-7, the light window 26 can be formed from a hole in the absorbent layer 20 with a continuous sealing layer 18 in order to keep the treatment fluid absorbed in the contact layer 16 electrically isolated from the lights 14. The sealing layer 18 can be a film or membrane that is impermeable to the treatment fluid.

In some embodiments, where the lights 14 have been waterproofed, the contact layer 16 may not include a sealing layer 18 and the light windows 26 can be holes through the entire contact layer.

In some embodiments, as shown in FIGS. 3 and 4, the backing layer 12 comprises a liquid impermeable backing layer 24. In the embodiment of FIG. 3, the support layer 22 on which the plurality of lights 14 are formed is attached to the backing layer 24. In the embodiment of FIG. 4, the support layer and the backing layer 24 are the same material. As shown in FIGS. 1 and 2, the contact layer comprises a sealing layer 18 comprising a liquid impermeable material and an absorbent layer 20 adapted for retaining the treatment fluid. As evident from FIGS. 1-4, the backing layer 12 and the sealing layer 18 form a waterproof enclosure around the plurality of lights.

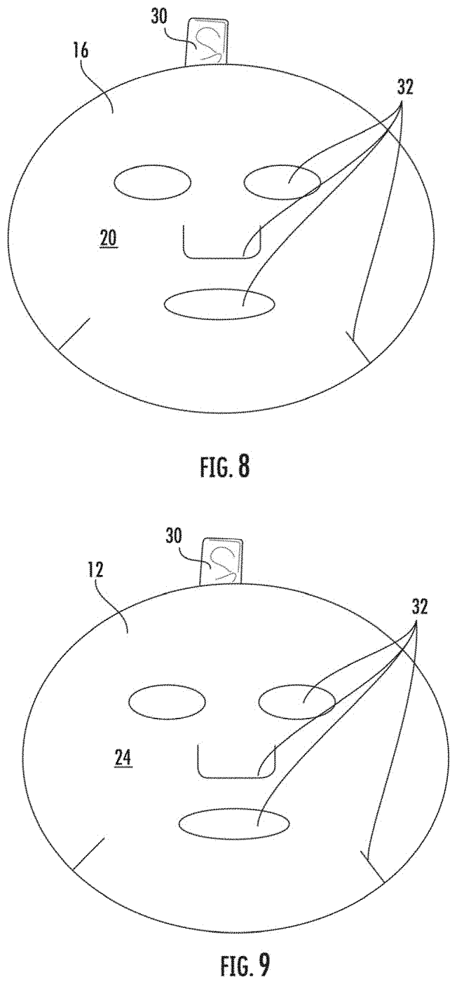

The absorbent layer 20 can be in immediate contact with the wearer's tissue. When the device is a mask, as shown in FIGS. 8 and 9, the phototherapy device 10, including the fabric layer will likely have wearer openings 32 (e.g., eye and mouth holes, a slit for the nose, and edge slits to conform to the jaw line). In some embodiments, the absorbent layer 20 will have holes for light to reach the user's face. In some embodiments, the absorbent layer 20 may assist in diffusing the light to the user's face (or other underlying tissue), with a light transmission rate greater than 10%. The absorbent layer 20 can be made out of a material including, but not limited to, cotton, pulp, cellulose (e.g., rayon), polymer, microfiber, cupra, Tencel, hydrogel, biocellulose, foil, clay/charcoal, ecoderma, sponge, foam, biologics (e.g., gelatin, cellulose, collagen, derivatives thereof), etc.

In some embodiments, this absorbent layer 20 will be white to assist in light reflection and transmission. In some embodiments, the absorbent layer 20 can be translucent or partially translucent, soft, porous, breathable, moldable, comfortable, absorbent, non-irritating, and thin.

[96] In some embodiments, as shown in FIGS. 3-4, the plurality of lights are formed on one or more substrates 22. In some embodiments, the substrate 22 can be a flexible printed circuit board (PCB) or another thin, soft, flexible material. Examples include, but are not limited to, paper, plastic, cloth, cotton, biologics (e.g., gelatin, cellulose, collagen, derivatives thereof, etc.), polymers, etc. In some embodiments, the one or more substrates 22 can be attached to a backing layer 24. In some embodiments, the one or more substrates 22 function as the backing layer 24. In some embodiments, each of the plurality of lights can be a light-emitting diode (LED), a laser diode, or another light source.

In some embodiments, such as that shown in FIG. 5, the backing layer 12 can be a PCB that is essentially the same shape as the contact layer 16. In such embodiments, the backing layer 12 and contact layer 16 can be attached via an edge seal 36 between the substrate 12, 22, 24 and the backing layer 12.

In some embodiments, such as that shown in FIG. 6, the strips of PCB to which the lights 14 are attached are discontinuous and smaller than the shape of the contact layer 16. These PCB strips can be individually secured to the contact layer 16.

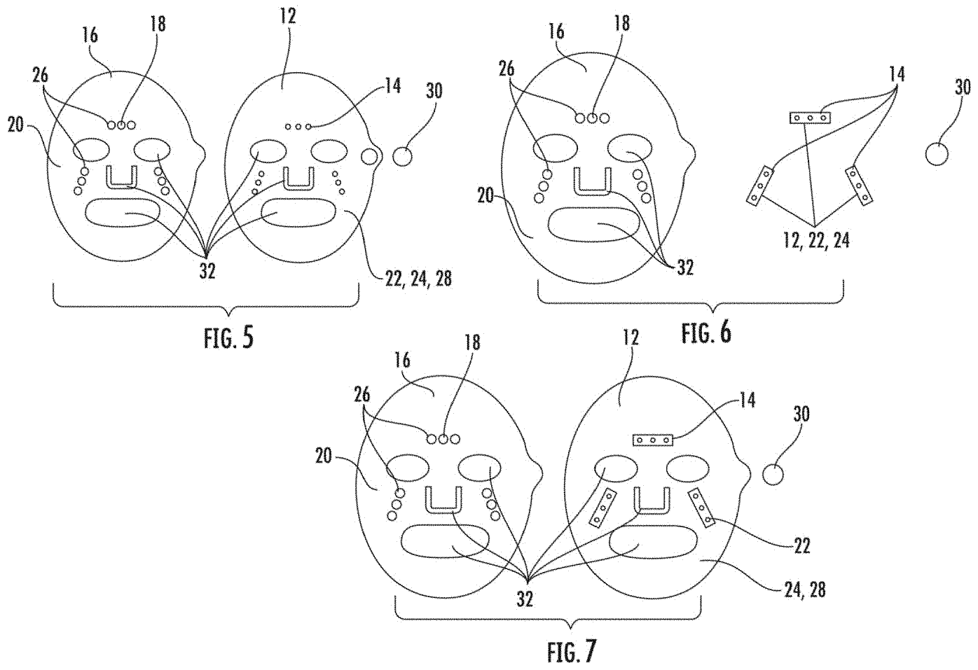

In some embodiments, such as that shown in FIG. 7, individual strips to which the lights 14 are attached are attached to the backing layer 24, which is essentially the same shape as the contact layer 16. In such embodiments, the backing layer 12 and contact layer 16 can be attached via an edge seal 36 between the backing layer 12, 24 and the backing layer 12.

In some embodiments, the phototherapy device 10 includes at least one flex PCB. Each of the PCBs can have a thickness ranging from 0.065 mm to 2.5 mm to assist in the conformity and flexibility of the phototherapy device 10. In some embodiments, the PCB/PCBs can include 1 to 4 copper layers.

In some embodiments, the phototherapy device 10 will include an array of LEDs, either all arranged in series or arranged in series parallel. In another embodiment, these LEDs will all be arranged in parallel. In some embodiments, the phototherapy device 10 will include an LED driver to ensure battery level does not affect light output. In other embodiments, the LEDs will all be driven using passive components (resistors, inductors, and capacitors).

In some embodiments, the phototherapy device 10 can include a timing circuit to pulse the array of LEDs. In some embodiments, an integrated circuit will be used to control the pulsing feature. In another embodiment, only passive components will be used to control this pulsing feature.

In some embodiments, the plurality of lights 14, either individually or in groups such as in an array, can be encased in a capsule 15a, as shown in FIGS. 32 and 37A, or attached to a backing 15b, as shown in FIGS. 34 and 37B. In some embodiments, the capsule or backing 15(a or b) provide a discrete water-tight volume 34 for the lights 14. In some embodiments, the capsule 15a or backing 15b can include a reflective layer 28 adapted for reflecting the treatment wavelength toward the contact layer 16.

In some embodiments as shown in FIG. 37A, a capsule (or pod) 15, can encase one or more light sources 14, such as LEDs. In some embodiments as shown in FIG. 37B, the one or more light sources 14, such as LEDs, can be encased within or otherwise mounted on a backing 15b. The lights 14 within the capsule or on the backing 15 can be the same wavelength, or can include two or more different wavelengths. In some embodiments, the capsule or backing 15 can also include a control that enables the wearer to select settings for the lights 14, for example, to select the wavelength and/or which lights to turn on, emission intensities, applications (continuous vs. pulsed), time durations, etc.

In some embodiments, the capsule 15a, backing 15b, or backing layer 12 can also allow for diffusion of the light so the entire side of the capsule 15a, backing 15b, or backing layer 12 that is facing or in contact with the tissue of a wearer, or portions of the side that are greater than the surface area of the light sources 14 themselves, can deliver light. For example in some embodiments, this could be achieved with light piping similar to that of a tv backlight, or in other embodiments, through a plastic or coating that facilitates high-transmission diffusion.

In some embodiments, the capsule 15a, backing 15b, or backing layer 12 can also include a power source 30, for example a battery (e.g. rechargeable or not), a capacitor (e.g. capacitors, supercapacitors, ultracapacitors, etc.) or another method of powering the plurality of light sources 14 and/or other components, such as connections to or for an external power supply (e.g. external battery, external capacitor, wired to wall power, etc.) to power the light sources or to charge/recharge the power source.

In some embodiments, the capsule 15a, backing 15b, or backing layer 12 can also include additional electronics, such as communications modules (e.g. Bluetooth, Wi-Fi, etc.), cameras, magnifiers, sensors (e.g. for pigment, moisture, color, etc), timers, on/off modules, etc. In some embodiments, the phototherapy device 10 can independently, or by communicating with a computer, tablet, mobile phone, or similar remote device evaluate and customize phototherapy treatments based on information collected through such additional electronics. In some embodiments, the phototherapy device 10 can be used in association with another device or devices to collect such additional information and evaluate the tissue for properties such as moisture, lines, pores, oil, pigment, elasticity, color, etc.

In some embodiments, the capsule or backing 15 can form a water-tight volume 34 to electrically isolate the light sources 14 from any treatment fluid retained by the contact layer 16. In some embodiments, a back surface of the contact layer 16 can include a sealing layer of a liquid impermeable material to electrically isolate the light sources 14 from any treatment fluid retained by the contact layer 16.

In some embodiments, the plurality of light sources 14 (including on a capsule or backing for the light sources) can also include one or more retaining members 33 adapted to engage with corresponding retaining members 13 on the backing or contact layer 12, 16 or through the backing or contact layer 12, 16. For example, retaining members 33 and/or 13 can include magnets, ferrous materials (e.g. ferrous sheets, patches, tapes, coatings, particles, etc.), adhesives, hook and loop, snaps, hooks, elastic bands or loops, clips, suction fasteners or other suitable fasteners.

In some embodiments, the plurality of lights 14 can be provided together with the phototherapy device 10 or separately from the phototherapy device 10 for attachment to the phototherapy device 10 by a wearer. For example, capsules 15a, backings 15b, or backing layers 12 containing the light sources 14 can be provided together or separately from the device 10.

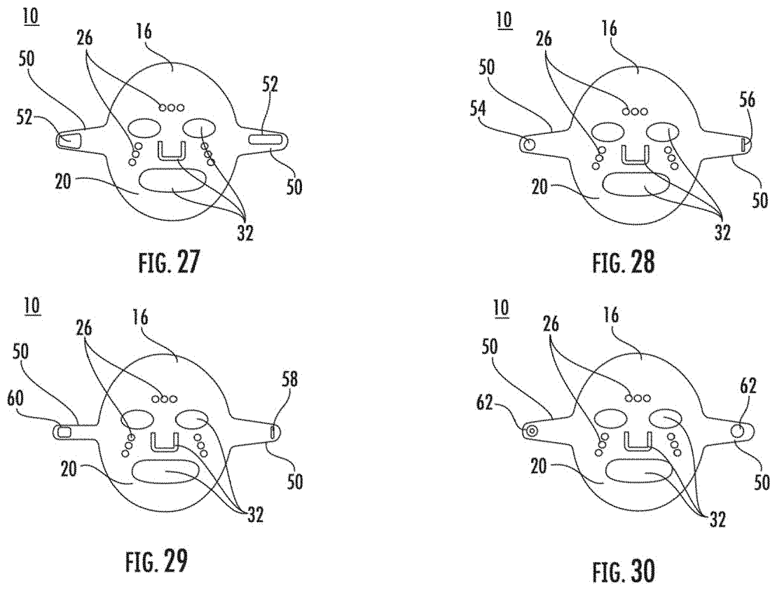

In some embodiments, the phototherapy device 10 can include one or more attachment features to facilitate attachment to the wearer. Examples of attachment features includes straps, bands, hook and loop material, frames, adhesive, etc. For example, FIGS. 27-30 show phototherapy devices 10 in the form of masks that includes two straps 50 for securing the phototherapy device 10 to the wearer's head. As shown in FIG. 27, in some embodiments the straps 50 can be releasably attached using hook and look material 52. As shown in FIG. 28, in some embodiments the straps 50 can be releasably attached using a button 54 and a button hole 56. As shown in FIG. 29, in some embodiments the straps 50 can be releasably attached using a slot 58 adapted for receiving the opposing strap 50, which includes an adhesive patch 60. As shown in FIG. 30, in some embodiments the straps 50 can be releasably attached using a snap connector 62. In some embodiments, the attachment features (e.g. straps or bands) can include fabric, elastic, plastics, or other suitable materials. In some embodiments, the attachment features can be adapted to removaby attach to the ears, around the head, hands, neck, breasts, feet, abdomen, groin, or other applicable body part of the wearer. The attachment features can be provided on the contact layer 16 and/or on any of the backing layer 12, capsules or backings 15.

In some embodiments, the treatment wavelength can include ultraviolet light, visible light, infrared light, or combinations thereof. In some embodiments, the treatment wavelength is blue light in the range of 400 to 490 nm and/or red light in the range of 620 to 750 nm. Blue light (e.g., 400 to 490 nm) can be used to treat conditions including, but not limited to acne (e.g., P. acnes) or other infections. Among other uses, red light (e.g., 620-750 nm) can be used to enhance blood flow, reduce inflammation, facilitate rejuvenation, repair, discoloration, healing, and reduce scarring Near infrared (NIR) radiation (e.g., 700-1,400 nm) can be used for tissue rejuvenation (repairing tissue damage), increased blood supply, increased collagen deposition, and decrease inflammation, facilitate rejuvenation, repair, discoloration, healing, and reduce scarring.

In addition, ultraviolet (UV) light can be used to facilitate wound healing and reduce/eliminate infection. UV light includes UVA (e.g., 315-400 nm), UVB (e.g., 280-315 nm), and UVC (e.g., 100-280 nm). In some embodiments, the treatment wavelength can be orange light or green light.

In some embodiments, the phototherapy device 10 includes a transmitter or transceiver 42 for sending and/or receiving information to a remote device. In some embodiments, the transceiver 42 can be adapted for communicating via Bluetooth, Wi-Fi, or other communication protocols.

In some embodiments, the phototherapy device 10 includes a power source 30 for powering the plurality of lights 14, the transceiver 42, or both. In some embodiments, the power source 30 can be battery (e.g., a button battery). In some embodiments, this power source 30 can be rechargeable. In some embodiments, this power source 30 can be directly attached to the plurality of lights 14 using a surface mount device or affixed component. In others, the power source 30 will be connected to the PCB using conductive wire or small conductive tabs. In some embodiments, the power source can be a power cord that can be connected to a separate battery pack or a power socket.

In some embodiments, the phototherapy device 10 includes a treatment fluid absorbed within, or to be absorbed within, the contact layer 16. Examples of active ingredients in the treatment fluid include, but are not limited to:
1. Antiaging—retinol, stem cells, primrose, grape seed extract, vitamin C, peptides, AHA's/alpha hydroxy acids, growth factors
2. Brightening—green tea, licorice, grapefruit, vitamin C, vitamin E, kojic acid, ferulic acid, peptides
3. Acne—salicylic acid, AHA, BHA, glycolic acid, citric acid, zinc, tea tree, green tea, cucumber, thyme
4. Hydrating—glycerin, argan oil, hyaluronic acid, aloe vera, ceramides, rosehip oil, sea kelp, jojoba, vitamin E
5. Exfoliating—AHA's/glycolic acid, lactic acid, retinol, fruit/plant enzymes, citric acid
6. Repair/Renew—retinol, ceramides, resveratrol, antioxidants, omega 3′s, essential oils, seaweed, noni, green tea.

In some embodiments, the phototherapy device 10 is adapted so that, when the treatment fluid is absorbed by the contact layer 16 and the contact layer 16 contacts tissue of a wearer, the phototherapy device 10 will remain in place even when the tissue surface is at an incline of at least 35° along a sagittal axis of the wearer. In some embodiments, the phototherapy device will remain in place when the tissue surface is at an incline of at least 45°, or of at least 55°, or of at least 60°, or at least 65°, or at least 70° along a sagittal axis of the wearer. In some embodiments, the phototherapy device will remain in place when the tissue surface is at an incline of at least 90°, or of at least 120° along a sagittal axis of the wearer. For example, a wearer can bend in a range of motion in various directions while the phototherapy device is applied and the device remains in place.

In some embodiments, as shown in FIGS. 11 and 12, the backing layer 12 and the contact layer 16 are coupled by an edge seal 36.

In some embodiments, as shown in FIG. 11, at least a portion of the backing layer 12 and the contact layer 16 are permanently coupled together (e.g., hot melt adhesive, thermal bonding, etc.) to form an edge seal 36. In some embodiments, an entire perimeter of the backing layer 12 and the contact layer 16 are permanently coupled together to form an edge seal 36.

In some embodiments, as shown in FIG. 12, at least a portion of the backing layer 12 and the contact layer 16 are releasably coupled together to form an edge seal 36. In some embodiments, an entire perimeter of the backing layer 12 and the contact layer 16 are releasably coupled together to form an edge seal 36. In some such embodiments, the contact layer 16 can wrap around and be secured to the backing layer 12 using a releasable seal material 38. In some embodiments, the releasable seal material 38 can be a material including, but not limited to, hook and loop material, releasable adhesive/tape, clips, glue, and magnets.

In some embodiments, the backing layer 12 can cover and/or be coupled to only a portion of the contact layer 16.

In some embodiments wherein the plurality of lights 14 are attached to a backing layer 12, the backing layer 12 can include attachment features (e.g. straps or bands) 50 as described herein that are adapted to removaby attach to the ears, head or other applicable body part of the wearer and to cover and secure the contact layer 16, or portions of the contact layer, therebetween when the attachment features 50 are coupled to the wearer.

In some embodiments, as shown in FIGS. 31 and 32, the backing layer 12 can form the one or more retaining members 13 adapted to hold the plurality of lights 14 and attached to the contact layer 16. In such embodiments, the retaining members 13 can comprise sleeves, pockets, cut out slits, straps, elastic or similar configurations coupled to the contact layer 16. In some embodiments, the retaining members 13 can include elastic materials or can include fasteners allowing the size of the retaining members to be adjusted.

In some embodiments, as shown in FIGS. 33, 34 and 39, the phototherapy device 10 can include retaining members 13 comprising fasteners coupled to the contact layer 16, such as magnets, ferrous materials, adhesives, hook and loop, snaps, hooks, elastic bands or loops, clips, suction fasteners, or other suitable fasteners for receiving a corresponding portion of the lights 14. In such embodiments, the backing layer 12 may or may not be included on the phototherapy device 10. For example, the retaining members 13 can be provided on capsules 15a, backings 15b, or otherwise coupled to the light sources 14.

The retaining members 13 can releasably hold the lights 14. In embodiments wherein the plurality of lights 14 are provided in capsules or on backings, the retaining members can releasbly hold the capsule or backing. For example, as shown in FIG. 32, the lights 14 can be removably inserted into retaining members 13 comprising a sleeve or pocket; or as shown in FIG. 34, the lights 14 can be removably attached to retaining members 14 comprising fasteners. In some embodiments, the capsule or backing for the lights 14 can include corresponding retaining members 33 for engaging with the retaining members 13, backing layer 12, and/or contact layer 16. FIG. 39 shows some exemplary types of retaining members 13 coupled to the contact layer 16, such as ferrous patches 13a or magnets 13b.

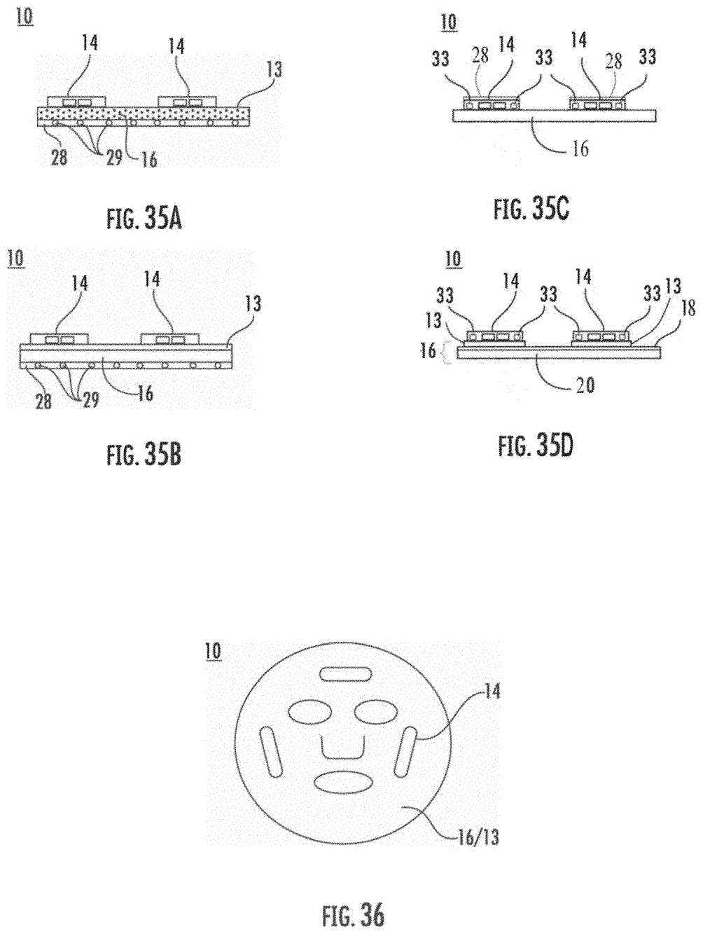

In some embodiments, as shown in FIGS. 35a, 35b, 39 and 41, the retaining member 13 can be integrated with the contact layer 16, such as impregnating, coating or covering the contact layer 16, or one or more portions of the contact layer 16, with the retaining member 13. For example in some embodiments, the contact layer 16 can be impregnated with magnetic material 13 (or 13d), and the plurality of lights 14 can include a corresponding magnetic material. In other embodiments, the contact layer 16 can be covered with a loop material, and the plurality of lights 14 can include a corresponding hook materials, or vice versa. In some embodiments, the retaining member 13 can cover, coat or be impregnated in substantially all of the surface area of the contact layer 16, or only a portion of the contact layer 16, or in locations corresponding to one or more treatment areas on the wearer when the device 10 is worn.

In some embodiments wherein the retaining members 13 are provided on the contact layer 16 and/or wherein the retaining members 33 are provided on the light sources 14 (for example on capsules or backings) as shown in FIG. 41, the plurality of lights 14 can be removably coupled to one side of the contact layer 16, and the other side of the contact layer 16 is adapted to contact the tissue of the wearer.

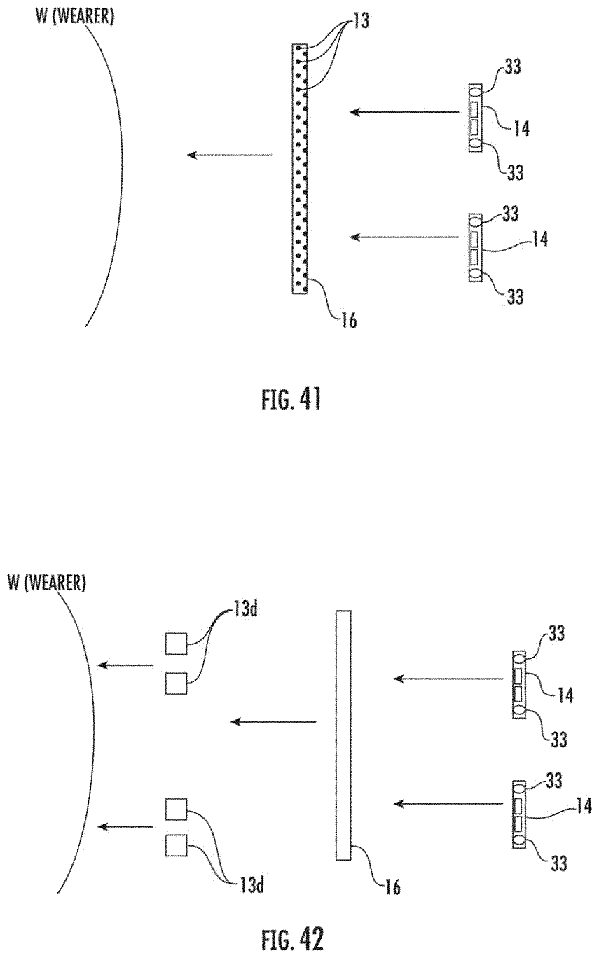

In some embodiments, as shown in FIG. 42, the phototherapy device 10 can include retaining members 13d adapted to be removably coupled to a user's skin on one side of the retaining member 13d (e.g. by adhesives or other skin-suitable materials) and adapted to removably couple with retaining members 33 for the lights 14 (e.g. on the capsules or backings 15) securing the contact layer 16 therebetween when the retaining members 13d and 33 are coupled.

In some embodiments, as shown in FIGS. 32, 34 and 39, the retaining members 13 can be coupled to the contact layer 16 in locations corresponding to one or more treatment areas on the wearer when the device 10 is worn. For example, in embodiments adapted to be worn on and treat the face, locations on the sheet mask 16 may generally correspond to one or more of the forehead, cheeks, under eye, chin, or nose. The lights 14 can be selectively inserted or attached in locations corresponding to a desired treatment area.

In embodiments providing retaining members 13 in multiple locations corresponding to one or more treatment areas on the wearer when the device 10 is worn or covering substantially all of the surface area of the contact layer 16, for example as shown in FIG. 36 and FIG. 39, the location and placement of the lights 14 can be selected by the wearer, as desired.

In some embodiments, the phototherapy device 10 to be customized to treat different areas and conditions. For example, the device 10 can be customized with the selective placement of lights 14 and the selection from a plurality of lights 14 with different features, such as treatment wavelengths, timers, pulsing, or other features.

In some embodiments, the contact layer 16 is adapted to contact a wearer of the phototherapy device 10 and the contact layer 16 is adapted to deliver treatment fluid retained therein to the wearer of the phototherapy device 10.

In some embodiments, the phototherapy device 10 can be adapted such that at least one of the following is true regarding the plurality of lights 14: (i) they can emit at more than one target wavelength, (ii) they emit at more than one target intensity, (iii) they can emit either continuously or pulsed intermittently, and (iv) they can emit for more than one pre-programmed duration. In some embodiments, the pre-programmed duration can range from 2 seconds to 180 minutes, or 1 minute to 120 minutes, or 3 minutes to 60 minutes, or 5 minutes to 45 minutes, or 10 minutes to 20 minutes, or 10 minutes to 30 minutes, or 15 minutes to 25 minutes, or 15 minutes to 30 minutes or 20 minutes to 30 minutes. The treatments can be performed twice a day, once a day, once every second day, twice a week, once a week, or any other desirable frequency.

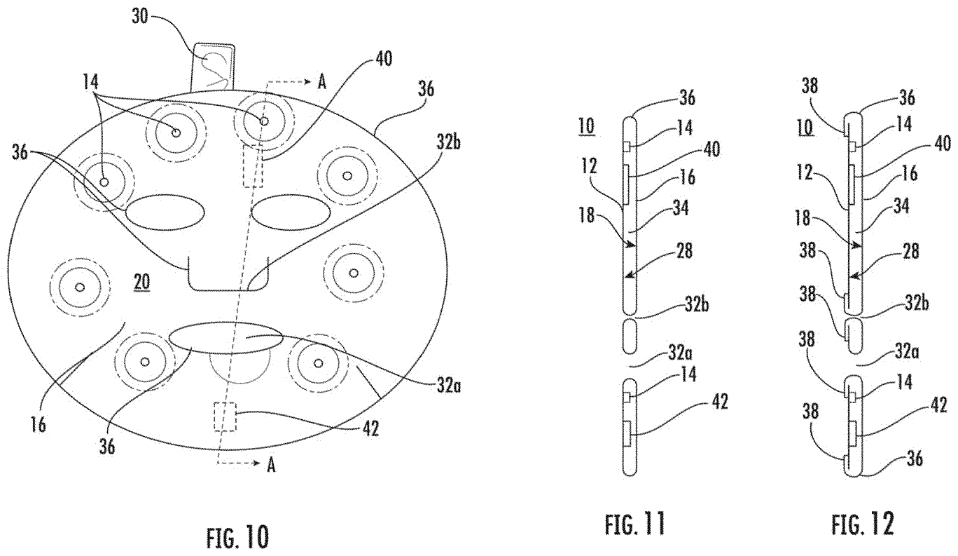

In some embodiments, as shown in FIGS. 10-12, the phototherapy device 10 can include a control 40 that enables the wearer to adjust at least one of options (i)-(iv). In some embodiments, the control 40 can be one or more buttons or switches. In some embodiments, control 40 includes at least one processing device, such as a central processing unit (CPU) or general processing unit (GPU). The selection(s) can be made by a remote device (e.g., a mobile device, such as a cellular phone, tablet, personal computer, etc.) and transmitted to the control 40 of the phototherapy device 10 via the transmitter or transceiver 42.

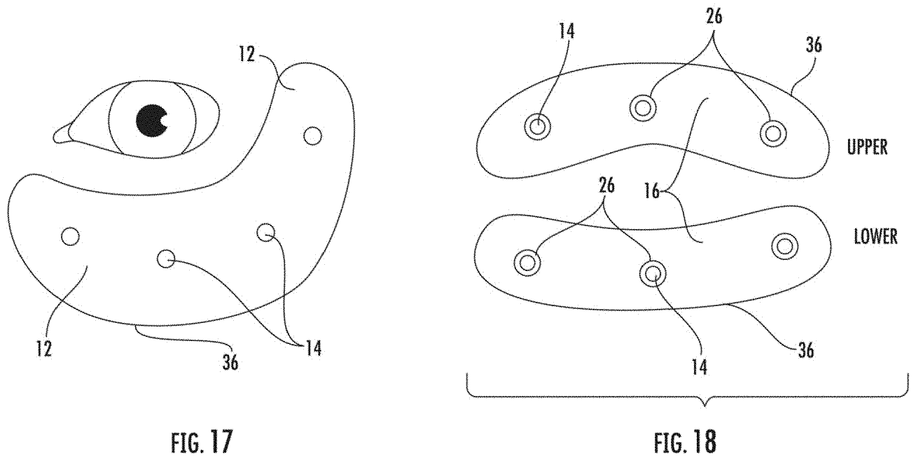

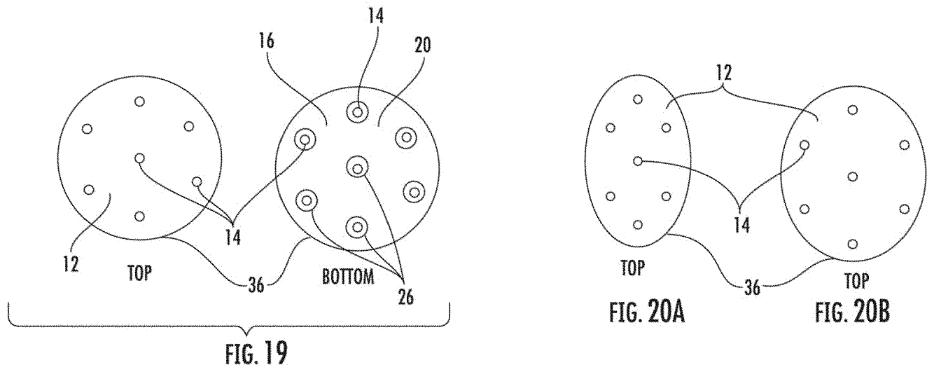

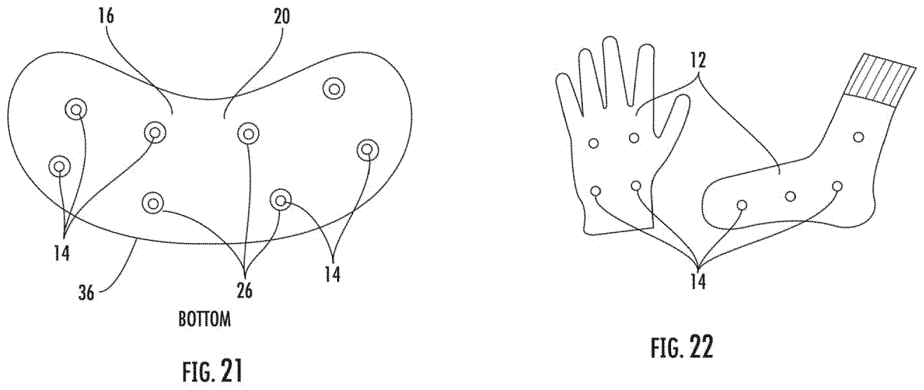

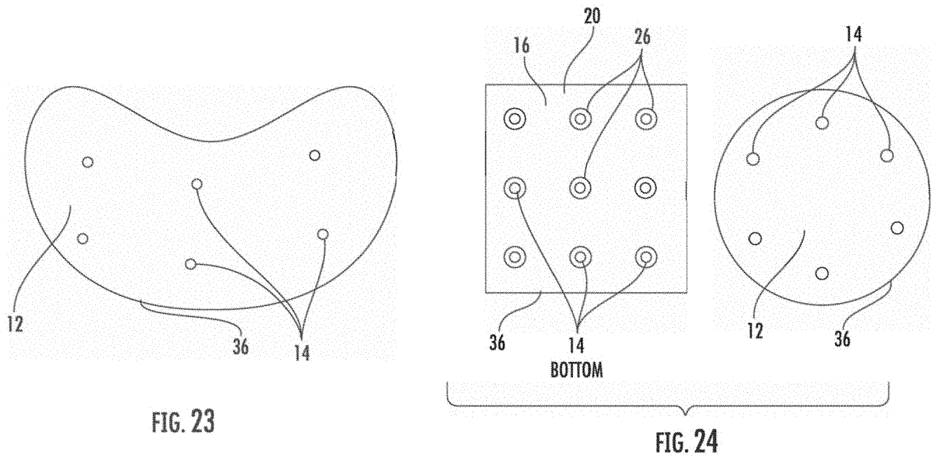

In some embodiments, the phototherapy device 10 is adapted to be worn on and treat a part of the body such as the face, under the eye, lips, breasts, décolletage, and groin. For example, FIG. 17 shows a top view of an arcuate phototherapy device 10 adapted to be treat a wearer's eye, FIG. 18 shows a bottom view (tissue contacting side) of two arcuate phototherapy devices 10 adapted to treat a wearer's upper and lower lips, FIG. 19 shows a top and bottom view of a generally round phototherapy device 10 adapted to treat a wearer's breast, FIGS. 20A and 20B show top views of a phototherapy device 10 adapted to treat a wearer's genitals (female and male, respectively), FIG. 21 shows a bottom view of a generally arcuate phototherapy device 10 adapted to treat a wearer's neck, FIG. 22 shows perspective views of a phototherapy device 10 in the form of a sock and a glove adapted to treat a wearer's feet and hands, respectively, and FIG. 23 shows a top view of a phototherapy device 10 adapted to treat a wearer's décolletage. FIG. 24 shows a bottom view (left) and a top view (right) phototherapy devices 10 that can be used to treat a variety of parts of the body, but that are not shaped for a specific body part.

Because of the pliability of the phototherapy devices 10 described herein, the phototherapy devices 10 described herein can be used for nearly any body part they are large enough to cover. One exception is face, which generally requires one or more wearer openings 32 over the mouth and/or nose to allow for breathing and, optionally, sight. Examples of phototherapy devices 10 adapted to treat the face are shown in FIGS. 5-16, 32, 34, 36 and 39.

In some embodiments, the treatment liquid can be a serum—either included with the mask or of the consumers own choosing. Examples of a serum are compositions, generally fluids, that can be absorbed by the contact layer and contacted with the patient's skin/tissues in order to complement or enhance the efficacy of the light treatment. As used herein, concentrated is intended to refer to an active ingredient that is present in high proportion relative to other substances.

The treatment liquid can be easily absorbed oil or water based liquids that are applied to the tissue. The treatment liquid can be a concentrated formula that allows targeted treatments and better absorption. These results will be magnified when used as part of the phototherapy device 10. The treatment liquid can be gel like or have a powdered base.

Examples of treatment liquid (e.g., serum) categories include, but are not limited to, anti-aging, acne, skin brightening, hydrating, exfoliating, reparative/renewing, and treatment of various tissue disorders, including wound healing, scar reduction, etc. In some embodiments, the treatment liquid comprises an ingredient selected from the group consisting of retinol, salicylic acid, zinc, glucolics, tea tree oil, vitamin C, kojic acid, peptides, growth factors, and antioxidants, niacinamide, polyphenols, glutathione, flavonoids, stem cells, primrose, grape seed extract, vitamin E, co-Q10, alpha hydroxy acids, beta hydroxy acids, licorice, grapefruit, ferulic acid, glycolic acid, citric acid, zinc, green tea, cucumber, thyme, glycerin, argan oil, hyaluronic acid, aloe vera, ceramides, rosehip oil, sea kelp, jojoba, lactic acid, fruit/plant enzymes, resveratrol, antioxidants, omega 3′s, essential oils, seaweed, and noni.

The treatment liquid can include additional active ingredients, as well as, cosmetically acceptable carriers, fillers, thickeners, and inactive ingredients. In some of the methods described herein, use of the phototherapy device 10 can be preceded by or followed by appropriate tissue care regimens, including application of cleansers, toners, sunscreens, moisturizers, or any other type of tissue treatment.

As discussed above, in some embodiments, the treatment liquid can also include reflective particles 29 as an additional or alternative reflective material 28.

In some embodiments, the treatment fluid can be provided together with the phototherapy device 10 or separately from the phototherapy device 10 for application to the phototherapy device 10 by a wearer.

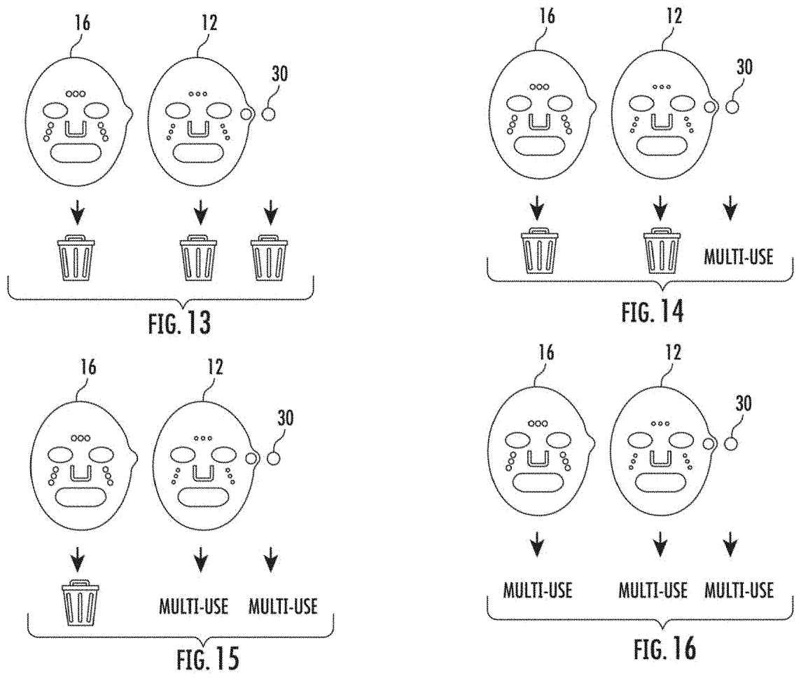

As shown in FIGS. 13-16, the phototherapy device 10 described herein can be disposable (FIG. 13) or part or all of the phototherapy device 10 can be reused (FIGS. 14-16. For example, as shown in FIG. 14, the battery can be reused in some embodiments. In other embodiments, as shown in FIG. 15, the backing layer (e.g., including the plurality of lights 14) and battery can be reused. In other embodiments, as shown in FIGS. 37A-B, the plurality of lights 14 (e.g. lights 14 in a capsule 15a or on a backing 16b, together with any other electronics included in the capsule or backing) can be reused. In some embodiments, the contact layer 16 can be disposable. In yet other embodiments, as shown in FIG. 16, the entire phototherapy device 10 can be reused.

As will be understood, the embodiments of FIG. 15 will require that the backing layer 12 and the contact layer 16 are releasably coupled. Similarly, in instances where the electronics (e.g., plurality of lights 14, power source 30 and, where present, controls 40 and transceiver 42) are reusable but the backing layer 12 and/or contact layer 16 are not, the electronics can be removably attached to the backing layer 12 and/or contact layer 16. In such embodiments, the electronics (whether in a capsule or backing 15 or not) can be releasably attached using a system including, but not limited to, retaining members 13 and/or 33 as described herein.

As a result of the unique design of the phototherapy device 10, it is highly foldable. The phototherapy device 10 should be easily foldable to fit in smaller packaging. This necessitates that the device should be thin, soft, flexible, pliant, and durable for comfort of treatment and adequate shelf life. The device is easily foldable to half its original surface area or less and can be stored folded in half or in thirds or in quarters, then be unfolded and used. In some embodiments, the device can be folded in an irregular and/or asymmetric pattern.

As described above, the phototherapy device 10 should also be highly conformable. Due to the wet contact layer with serum, the device should easily conform to the wearer's anatomy. This is improved by shape, thinness, design (additional slits/cuts) to allow close adherence. The phototherapy device 10 will conform to each individual's unique anatomical shape.

The closer the light source is to the target, the stronger the “dose”/photon delivery. Thus, the highly conformable nature of the phototherapy device 10 allows the closest delivery in a noninvasive delivery system (i.e., the most efficient photon delivery system possible).

As described above, the phototherapy device 10 is also highly customizable for targeted application and treatment of a broader range of conditions. With the selective placement and removable attachment of lights 14 and/or the selection from a plurality of lights 14 with different features, such as treatment wavelengths, timers, pulsing, or other features, the device 10 can be configured by the user for more targeted treatment of the user's specific conditions and re-configured by the user for targeted treatment of various other conditions.

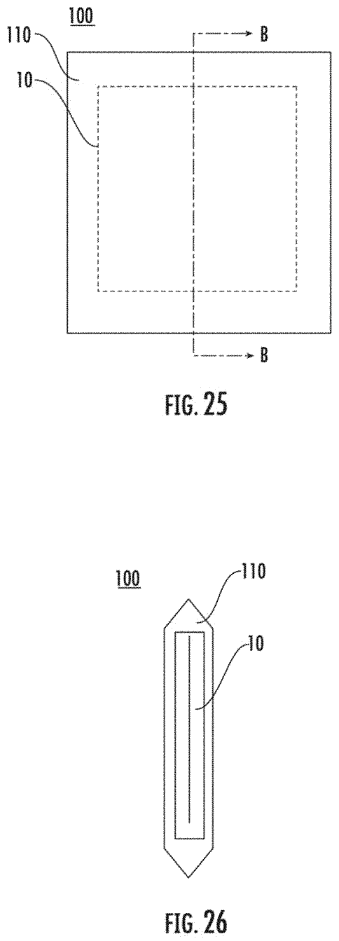

In a related embodiment, as shown in FIGS. 25 and 26, a tissue treatment package 100 is provided. The tissue treatment package 100 can include a folded phototherapy device 10 as claimed herein and a sealed package 110, where the folded phototherapy device 10 is contained within the sealed package 100. In addition, (i) a liquid treatment is absorbed in the contact layer, or (ii) the sealed package contains a liquid treatment to be applied to the contact layer, or (iii) both (i) and (ii). In some embodiments of the tissue treatment package 100, the treatment liquid is absorbed in the contact layer 16, and the sealed package 110 is adapted to retain the treatment liquid therein. In other words, the sealed package 110 can be impermeable to the treatment liquid.

In some embodiments, the sealed package 110 can be a thin foil package that is torn open. In some embodiments, the sealed package 110 can be 3-6 inches in width and 3-8 inches in length, and several mm thick. As the phototherapy device 10 is folded, the sealed packaging 110 is significantly smaller than the anatomical structure/body part treated by the phototherapy device 10.

In one embodiment, the sealed package 110 includes the entire phototherapy device 10, all layers and electronics in one sealed space. In another, the electronics (e.g. lights 14 etc., whether or not in capsules, backings or a backing layer) will be packaged, and the contact layer will be separately packaged. In another embodiment, the packaging will include an electronics layer or one or more electronics capsules/backings to be reused, and include single or multiple soaked or unsoaked contact layers to be attached after opening. In another embodiment, the contact layer(s) would be “soaking” in the serum/solution. In another embodiment, the serum module would be opened and added to the contact layer, or alternatively, allow the user or wearer to select and add a serum/solution of their own choosing.

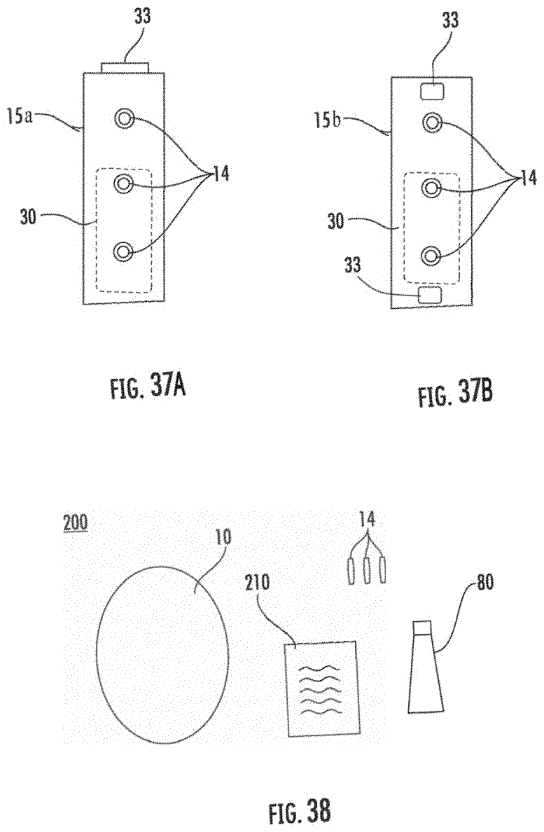

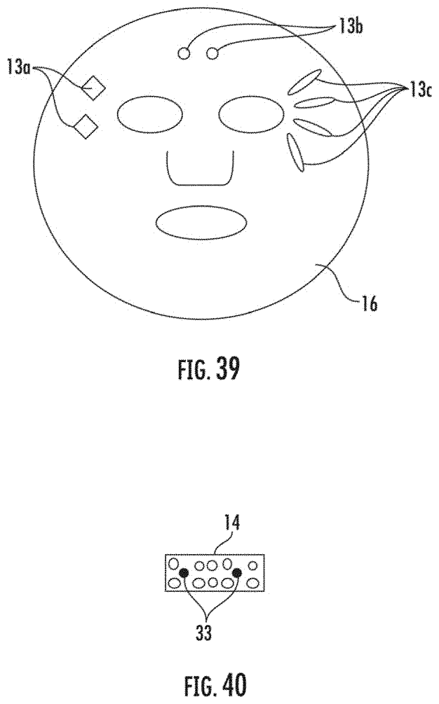

In another embodiment, a phototherapy kit 200 is provided. In some embodiments, as shown in FIG. 38, the kit 200 includes a phototherapy device 10 as described herein and one or more items selected from: a plurality of lights 14, a treatment fluid 80, and instructions 210 for carrying out a method of using the phototherapy device 10 and any variants thereof described herein. The instructions 210 can be in the form of printed material, a mobile app or a web app, an instructional video on recorded media (e.g., DVD) or via a website, or any other approach for communicating the intended use of the phototherapy device 10. For example, in some embodiments, the instructions 210 can provide information to assist a wearer on selection and placement of the plurality of lights 14.

In some embodiments, the kit 200 can include a plurality of the same lights 14. In other embodiments, the kit 200 can include a plurality of different lights 14. For example, the lights 14 can be provided in two or more different treatment wavelengths, emission intensities, applications (continuous vs. pulsed), or time durations. Selection of different combinations from a plurality of lights 14 and attachment to different locations on the device allows the phototherapy device 10 to be customized and used to treat a wide variety of conditions and areas.

In another embodiment, a method of treating tissue by applying an electromagnetic radiation to tissue using a phototherapy device 10 as described herein is provided. In some embodiments, the method includes applying the electromagnetic radiation in pulses. In some embodiments, the method includes applying electromagnetic radiation at a treatment wavelength in the range of 400 to 490 nm or 635 to 700 nm.

In some embodiments, the phototherapy device 10 requires activation by the user before or during application. For example, in some embodiments, the wearer will need to activate the phototherapy device using the controls 40. In some examples, this may include pressing a button or flipping a tactile switch. In another embodiment, capacitive touch sensing, or any other means, can be used to turn on the phototherapy device 10 once in contact with a wearer's tissue. In another embodiment, the device turns on once the wearer opens the packaging 110.

In some embodiments, the method includes receiving a selection of one or more of the following: (i) a target wavelength, (ii) an emission intensity, (iii) application of the treatment wavelength as either continuously or pulsed, and (iv) application of the treatment wavelength for one of at least two available durations, and applying the target radiation based on the selection that was received.

In another embodiment, a method of treating tissue by applying an electromagnetic radiation to tissue using a phototherapy device 10 as described herein is provided. In some embodiments, the method includes providing a phototherapy device 10 as described herein, providing a plurality of lights 14, and providing instructions on attachment of one or more of the plurality of lights 14 to the phototherapy device 10. For example in some embodiments, the method can include placing the contact layer on top of the wearer's skin and attaching the plurality of lights 14 on top of the contact layer in the desired or instructed anatomical positions. In some embodiments, the instructions include different selections and placement of lights 14 on the phototherapy device 10 to apply to different conditions.

In another embodiment, a method of treating tissue by applying an electromagnetic radiation to tissue using a phototherapy device 10 as described herein is provided. In some embodiments, the method includes providing a phototherapy device 10 or a phototherapy kit 200 as described herein, receiving information about the intended wearer, and providing use instructions on one or more of the following: (i) selection type of the plurality of lights 14, (ii) placement of the plurality of lights 14 on the device 10, (iii) selection of settings on the plurality of lights 14, (iv) selection of treatment fluid, (v) duration of application, and (vi) application frequency.

In some embodiments, the information can be received as a photograph or other image of the tissue to be treated, or as text, survey responses, or other data about the tissue to be treated. In some embodiments, the instructions can be provided via automated means, including without limitation, providing instructions via the mobile app, web app, or website in response to a user submission of information.

In some embodiments, the use instructions can be provided to and/or received by an intended wearer or other user via a mobile app, web app, or website. For example, instructions 210 may instruct the user to download an application (e.g., “App”) to a computing device, such as a computer, laptop, or mobile device (e.g., cellular phone, smartphone, etc.). The computing device is capable of executing the application, which facilitates communications with a server, such as a web server, over a communications network. The communications network can be, for example, a WiFi® network, a cellular network such as a 3GPP® network, a Bluetooth® network, a satellite network, a wireless local area network (LAN), a network utilizing radio-frequency (RF) communication protocols, a Near Field Communication (NFC) network, a wireless Metropolitan Area Network (MAN) connecting multiple wireless LANs, a wide area network (WAN), or any other suitable network. The communication network can provide access to, for example, the Internet.

Once downloaded and executed, the application may provide the use instructions to the user (e.g., via a display of the computing device). In some examples, the application may further instruct the user to take one or more images (e.g., with a camera of the computing device, or any other camera) of the tissue to be treated. The application may facilitate the transmission of the one or more images to the server. In some examples, a treating physician may view the images, and provide recommendations for placement of the lights to the intended wearer based on the images. For example, the treating physician may provide recommendations based on the presence of acne lesions (or other imperfections) as seen in the images. The treating physician may provide the recommendations to the server, and the server may transmit the recommendations to the computing device. The computing device may display the recommendations to the user via the application. In some examples, the application facilitates the capturing of images over time (e.g., periodically, such as weekly, monthly, etc.), and transmitting the images to the server for consideration by the treating physician. For example, the server may display the images to the treating physician, and the treating physician may decide to alter the treatment, or even stop the treatment, based on the images.

In some embodiments, the server is configured to apply one or more machine learning models (e.g., algorithms, computer vision models, etc.) to the received images to generate the recommendations. For example, the server may train a machine learning model with labeled image data that identifies the presence, or lack thereof, of lesions (or other imperfections) on various tissue types and/or areas (e.g., on any skin type and/or face, neck or décolletage, etc.), as well as treatment data (e.g., placement and/or orientation of lights on the phototherapy device for application to the tissue, selection type of the plurality of lights, a placement and/or orientation of the plurality of lights on the device, a placement and/or orientation of lights (including light capsules, etc.) directly or indirectly on tissue, selection of settings on the plurality of lights, selection of treatment fluid, duration of application, application frequency, etc.). Once trained, the server may validate the machine learning model during a validation period. Once one or more predetermined thresholds are met (e.g., accuracy metrics, etc.), the server may deploy the trained machine learning model to operate on the images received from a user.

The server may be configured to apply the trained machine learning model to the received images, and generate recommendations data. The recommendation data may identify, for the intended wearer or other user, one or more of the above-identified use instructions. For example, the recommendation data may identify a selection type of the plurality of lights, a placement and/or orientation of the plurality of lights on the device, a placement and/or orientation of lights (including light capsules, etc.) directly or indirectly on tissue, selection settings for the plurality of lights, selection of treatment fluid, a duration of application, or any other suitable information. The server may transmit the recommendation data to the computing device for presentation to the user.

In some embodiments, the application facilitates the scheduling of a meeting time with a treating physician. For example, the application may allow the intended wearer or other user to select a date and time to virtually meet with the treating physician. Once scheduled, the application may facilitate an audio and visual meeting with the treating physician, whereby the treating physician may provide use recommendations based on viewing video of a treatment area (e.g., face, etc.) of the intended wearer.

In some embodiments, the application facilitates the tracking of progress during a treatment period. For example, the application can remind the intended wearer or other user to capture one or more images of a treatment area (e.g., face, etc.) on a periodic basis (e.g., daily, weekly, etc.). The application can save the captured images (e.g., to a memory of the computing device), and can further archive the images based on a capture date and/or time. The application allows the intended wearer or other user to see and/or judge for themselves the device or treatment's success and, in some examples, determine their own regimen (e.g., increase application times, decrease application times, end treatment, etc.).

The foregoing is provided for purposes of illustrating, explaining, and describing embodiments of these disclosures. Modifications and adaptations to these embodiments will be apparent to those skilled in the art and may be made without departing from the scope or spirit of these disclosures.

What is claimed is:

1. A phototherapy device, comprising:
a plurality of light sources adapted for emitting at a treatment wavelength; and
a contact layer, wherein the contact layer is adapted to retain a treatment fluid;
wherein the plurality of light sources are electrically isolated from treatment fluid retained by the contact layer and the plurality of light sources are coupled to the contact layer.

2. The phototherapy device as claimed in claim 1, wherein the plurality of light sources are releasably coupled to the contact layer.

3. The phototherapy device as claimed in claim 1, wherein the contact layer comprises one or more retaining members for releasably engaging with one or more of the plurality of light sources.

4. The phototherapy device as claimed in claim 3, wherein the one or more retaining members are coupled to the contact layer in fixed locations corresponding to one or more treatment areas on an intended wearer.

5. The phototherapy device as claimed in claim 3, wherein the contact layer is impregnated, coated or covered with the one or more retaining members.

6. The phototherapy device as claimed in claim 1, wherein the plurality of light sources comprise one or more retaining members for releasably engaging with the contact layer.

7. The phototherapy device as claimed in claim 1, wherein the plurality of light sources are encased in one or more capsules or attached to one or more backings, and the capsule or backings comprise one or more retaining members for releasably engaging with the contact layer.

8. The phototherapy device as claimed in claim 7, wherein the capsule or backing comprises a reflective layer adapted for reflecting the treatment wavelength toward the contact layer.

9. The phototherapy device as claimed in claim 1, wherein the plurality of light sources are coupled to the contact layer by a backing layer;
wherein the plurality of light sources are attached to the backing layer and the backing layer is coupled to the contact layer; and
the phototherapy device is pliable.

10. The phototherapy device as claimed in claim 9, wherein the backing layer comprises a reflective layer adapted for reflecting the treatment wavelength toward the contact layer.

11. The phototherapy device as claimed in claim 1, wherein the contact layer is adapted for transmitting the treatment wavelength emitted by the plurality of light sources.

12. The phototherapy device as claimed in claim 1, wherein the treatment wavelength is in the range of 400 to 490 nm or 620 to 1700 nm.

13. The phototherapy device as claimed in claim 1, wherein each of the plurality of light sources is an LED.

14. The phototherapy device as claimed in claim 1, further comprising a treatment fluid absorbed within the contact layer.

15. The phototherapy device as claimed in claim 1, further comprising a communication component adapted for transmitting data and receiving data with a remote device.

16. A phototherapy kit, comprising:
the phototherapy device as claimed in claim 1; and
instructions for selection or placement of the plurality of lights, or for selection of settings for the plurality of lights, on phototherapy device.

17. The phototherapy kit as claimed in claim 16, further comprising a plurality of two or more different types of lights.

18. The phototherapy kit as claimed in claim 16, further comprising a sealed package,
wherein the phototherapy device or at least the contact layer of the phototherapy device is folded and contained within the sealed package, and
wherein (i) a liquid treatment is absorbed in the contact layer, or (ii) the sealed package contains a liquid treatment to be applied to the contact layer.

19. A method of treating tissue, comprising:
applying electromagnetic radiation to tissue using a phototherapy device as claimed in claim 1.

20. The method as claimed in claim 19, further comprising: receiving a selection of one or more of the following: (i) a target wavelength, (ii) an emission intensity, (iii) application of the treatment wavelength as either continuously or pulsed, and (iv) application of the treatment wavelength for one of at least two available durations.
